(19) United States
(12) Patent Application Publication
(10) Pub. No.: US 2003/0212118 A1
(43) Pub. Date: Nov. 13, 2003

(54) Treatment of eating disorders using carboxyalkylethers

(76) Inventors: Bruce Jeffrey Auerbach, Ann Arbor, MI (US); Donald Eugene Butler, Holland, MI (US)

Correspondence Address: David R Kurlandsky, Warner-Lambert Company, 2800 Plymouth Road, Ann Arbor, MI, 48105, US

(21) Appl. No.: 10/181,872

(22) PCT Filed: May 18, 2001
(86) PCT No.: PCT/US01/16334

(30) Foreign Application Priority Data
Jul. 14, 2000 (US) 60218399

(51) Int. Cl. A61K 31/41; A61K 31/225; A61K 31/19; A61K 31/11

(52) U.S. Cl. 514/381; 514/574; 514/547; 514/705

(57) ABSTRACT

arboxyalkcylethers are useful for treating eating disorders such as obesity.

### FIELD OF THE INVENTION

This invention concerns a method for treating eating disorders such as obesity and bulimia comprising administering a carboxyalkylether.

### BACKGROUND OF THE INVENTION

Eating disorders have become a major medical problem for which no suitable treatments are available. Many people, for example, suffer from conditions such as bulimia and anorexia. Obesity has become a major disease, particularly in the United States. While the etiology of obesity is not known, a factor common to all cases is necessarily the intake of amounts of food that supply more energy than the body uses. To date, the primary measure to correct this imbalance has been to reduce food consumption, ideally while at the same time increasing energy use. Persistent dietary restraint is essential, but is very difficult to achieve. Various sympathomimetic and related drugs that depress appetite have been used to make a low-calorie diet more tolerable. Such eating disorders generally are associated with psychological disturbances, and compulsive overeating is difficult to eradicate, even with psychiatric help.

Most of the drugs used to treat eating disorders are ineffective because of the inability to separate their central stimulant effects from their anorectic effects. The most commonly used anti-obesity agents are dextroamphetamine and methamphetamine. However, tolerance to these agents generally develops within a few weeks, and increased dosage is limited both by the peripheral actions that these drugs exert, and by such symptoms of central stimulation as nervousness and irritability. Several agents known as serotonin re-uptake inhibitors have been used, including fluoxetine hydrochloride (see U.S. Pat. No. 4,626,549) and fenfluramine. These agents cause significant central stimulation and generally are not well-tolerated at the dose levels required for anti-obesity and anti-bulimic effects.

We have now discovered that carboxyalkylethers are effective for treating eating disorders without causing CNS effects. These agents, such as those described in U.S. Pat. No. 5,648,387, which is incorporated herein by reference, do not operate as central stimulants, but instead interact with metabolism and are said to be insulin sensitizers and to effect cholesterol biosynthesis and metabolism. An object of this invention is to provide a method for treating eating disorders comprising administering a carboxyalkylether.

### SUMMARY OF THE INVENTION

This invention provides a method of treating eating disorders in a mammal comprising administering to a mammal in need of treatment an effective amount of a carboxyalkylether. The invention more particularly provides a method of treatment comprising administering a carboxyalkylether of Formula I

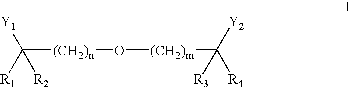

wherein

n and m independently are integers from 2 to 9;

R₁, R₂, R₃, and R₄ independently are C₁-C₆ alkyl, C₁-C₆ alkenyl, C₂-C₆ alkynyl, and R₁ and R₂ together with the carbon to which they are attached, and R₃ and R₃ together with the carbon to which they are attached, can complete a carbocyclic ring having from 3 to 6 carbons;

Y₁ and Y₂ independently are COOH, CHO, tetrazole, and COOR₅ where R₅ is C₁-C₆ alkyl, C₂-C₆ alkenyl, C₂-C₆ alkynyl; and

where the alkyl, alkenyl, and alkynyl groups may be substituted with one or two groups selected from halo, hydroxy, C₁-C₆ alkoxy, and phenyl.

Preferred compounds to be employed in this invention have the above formula wherein n and m are the same integer, and wherein R₁, R₂, R₃, and R4 each are alkyl.

Further preferred are compounds wherein Y₁ and Y₂ independently are COOH or COOR₅ where R₅ is alkyl.

The most preferred compounds to be employed have the Formula II

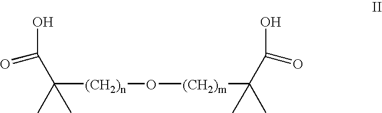

wherein n and m are each an integer selected from 2, 3, 4, or 5, ideally 4 or 5.

An especially preferred compound has the Formula III

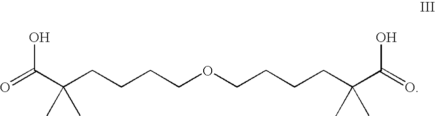

The monocalcium salt of the compound of Formula m is now known as CI-1027, and is currently being evaluated clinically for treating dyslipidemia.

### DETAILED DESCRIPTION OF THE INVENTION

As used herein, the term “carboxyalkylethers” means any compound having two alkyl groups linked through an oxygen atom, wherein one or both of the alkyl groups bears a substituent selected from a carboxylic acid group or salt thereof, a carboxylic acid ester, or a carboxylic acid miminic such as a tetrazole group. The alkyl groups can be any desired length, for example from 1 to 20 carbon atoms. As noted above, a preferred group of carboxyalkylethers for use in the method of this invention are those disclosed in U.S. Pat. No. 5,648,387. A particularly preferred carboxyalkylether for use herein is CI-1027, which is 6-(5-carboxy-5-methyl-hexyloxy)-2,2-diiethyl-hexanoic acid, calcium salt. This compound is also named 6,6′-oxybis-(2,2-dimethylhexanoic acid) mono-calcium salt. Other pharmaceutically acceptable salts can be employed, and these are described in U.S. Pat. No. 5,648,387.

The term “eating disorders” as used herein means any abnormal condition in which a mammal consumes or otherwise deals with food intake. The most common eating disorder encountered by humans is excessive food intake, resulting in an overweight or obese physical condition. Other eating disorders include bulimia, which is the abnormal increase in the sensation of hunger, also referred to as cynorexia Another typical eating disorder is anorexia, which is a lack or loss of the appetite for food, and is characterized by severe and prolonged inability or refusal to eat, sometimes accompanied by spontaneous or induced vomiting, extreme emaciation, amenorrhea, or other biological changes.

“Mammals” as used herein include humans, dogs, cattle, and sheep.

The ability of carboxyalkylethers to alter eating disorders has been established in several assays commonly used to measure the anti-obesity effects of chemical agents. The following example illustrates the anti-obesity effects of CI-1027 in such standard assays.

### EXAMPLE 1

Evaluation of CI-1027 in Female Obese Zucker Rats

Adult female obese Zucker rats weighing 265 to 268 g were obtained from Genetic Models, Inc. The animals were maintained on a diet of pelleted rodent chow (Ralston Purina) and were given water ad libitum. The animals were housed in cages in a temperature controlled room, with a 12-hour light/12-hour dark cycle (lights were turned on at 6 AM each day). CI-1027 was suspended in a vehicle of 1.5% carboxymethylcellulose containing 0.2% Tween-20. A control group of six rats were dosed daily with vehicle alone. One group of six test animals received daily oral dosing (by gavage) of 2.5 mL/kg suspensions of CI-1027 at 30 mg/kg active drug, while another group of six animals received daily oral dosing of 2.5 mL/kg of suspension at 100 mg/kg of CI-1027. The pelleted rodent chow was available ad libitun, except on Days 0, 7, 14, 21, 28, and 32, when food was removed from the cages at 7:30 AM, and the animals were weighed within 2 hours following removal of the food. The food trays were replaced in the cages after the weights of all animals were measured. The results of the test over 32 days are presented in Table 1.

TABLE 1
Effects of CI-1027 on Body Weight and Liver Weight in Female
Obese Zucker Rats

| | | | | | | | | Liver | |
|---|---|---|---|---|---|---|---|---|---|
| | | Body Weight | | | | | | Weight | Percent |
| Treatment | Dose | (g) | | | | | | (g) | Liver/Body |
| Group | mg/kg | Day 0 | Day 7 | Day 14 | Day 21 | Day 28 | Day 32 | Day 32 | Weight |
| Genetic Models | | | | | | | | | |
| Control | | 268 ± 4 | 299 ± 4 | 328 ± 4 | 350 ± 6 | 370 ± 5 | 379 ± 6 | 14.0 ± 0.4 | 3.70 ± 0.1 |
| CI-1027 | 30 | 265 ± 4 | 283 ± 4^{a} | 299 ± 4^{c} | 311 ± 3^{c} | 317 ± 4^{c} | 321 ± 4^{c} | 22.8 ± 1.0^{c} | 7.09 ± 0.3^{c} |
| | 100 | 265 ± 4 | 286 ± 3^{a} | 302 ± 4^{b} | 314 ± 4^{c} | 326 ± 5^{c} | 326 ± 5^{c} | 24.8 ± 1.3^{c} | 7.53 ± 0.3^{c} |

Data are mean ± SEM, (n = 6 rats/group).
Significantly different than respective control (^{a}< 0.05, ^{b}< 0.01, ^{c}<0.001)
Data were analyzed by two-sided unpaired t-Tests.

The data established that CI-1027 caused a significant reduction in weight in all treatment groups.

CI-1027 was also evaluated in leptin receptor deficient mice, and as expected, no significant effect on body weight or food consumption was observed.

For use as anti-obesity agents, the carboxyalkylethers will be formulated with common pharmaceutical excipients, diluents, and carriers as described in U.S. Pat. No. 5,648,387. The compounds can be administered orally or parenterally, with oral tablets, capsules, or solutions being preferred. The compound can also be administered as sustained or controlled slow release formulations for added convenience, for example as transdermal patches, osmotic pump tablets, and the like.

The carboxyailylethers will be administered at doses that are effective to produce an effect on the eating disorder to be prevented or treated. An “effective dose” will be any dose that results in a reduction in food consumption and/or a loss of weight. Typical doses will be from about 10 mg/kg/day to about 2000 mg/kg/day, with doses of about 150 mg/kg/day to about 600 mg/kg/day being preferred.

What is claimed is:

1. A method for treating eating disorders in a mammal comprising administering to the mammal in need of treatment an effective amount of a carboxyalkylether or pharmaceutically acceptable salt thereof.

2. A method for treating obesity comprising administering to a mammal in need of treatment an effective amount of a carboxyalkylether or pharmaceutically acceptable salt thereof.

3. A method for treating eating disorders in a mammal comprising administering an effective amount of a compound of Formula I 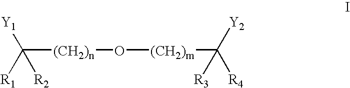
wherein
n and m independently are integers from 2 to 9;
R₁, R₂, R₃, and R₄ independently are C₁-C₆ alkyl, C₁-C₆ alkenyl, C₂-C₆ alkynyl, and R₁ and R₂ together with the carbon to which they are attached, and R₃ and R₃ together with the carbon to which they are attached, can complete a carbocyclic ring having from 3 to 6 carbons;
Y₁ and Y₂ independently are COOH, CHO, tetrazole, and COOR₅ where R₅ is C₁-C₆ allyl, C₂-C₆ alkenyl, C₂-C₆ alkynyl; and
where the alkyl, alkenyl, and alkynyl groups may be substituted with one or two groups selected from halo, hydroxy, C₁-C₆ alkoxy, and phenyl.

4. A method according to claim 3 wherein in the compound administered, R₁, R₂, R₃, and R₄ each are alkyl.

5. A method according to claim 4 wherein in the compound administered, Y₁ and Y₂ independently are COOH or COOR₅.

6. A method for treating eating disorders in a mammal comprising administering an effective amount of 6-(5-carboxy-5-methyl-hexyloxy)-2,2-dimethyl-hexanoic acid, or a pharmaceutically acceptable salt thereof.

7. A method for treating obesity in a mammal comprising administering an anti-obesity effective amount of 6-(5-carboxy-5-methyl-hexyloxy)-2,2-dimethyl-hexanoic acid, or a pharmaceutically acceptable salt thereof.
